(19) United States
(12) Patent Application Publication
(10) Pub. No.: US 2026/0325068 A1
(43) Pub. Date: Oct. 22, 2026

(54) SYSTEM FOR EVALUATING THERAPEUTIC EFFECT OF MENTAL ILLNESS AND/OR NEURODEGENERATIVE DISEASE

(71) Applicant: GUANGDONG ARTIFICIAL INTELLIGENCE AND DIGITAL ECONOMY LABORATORY (GUANGZHOU), Guangdong (CN)

(72) Inventors: Xinhong ZHU, Guangdong (CN); Minzhen ZHU, Guangdong (CN); Huanyu LI, Guangdong (CN)

(73) Assignee: GUANGDONG ARTIFICIAL INTELLIGENCE AND DIGITAL ECONOMY LABORATORY (GUANGZHOU), Guangdong (CN)

(21) Appl. No.: 18/993,571

(22) PCT Filed: Mar. 22, 2023
(86) PCT No.: PCT/CN2023/083089

§ 371 Date: Jan. 12, 2025

(30) Foreign Application Priority Data
Jul. 12, 2022 (CN) 202210816075.1

(51) Int. Cl. A61B 5/00

(52) U.S. Cl. CPC A61B 5/4848; A61B 2503/40; A61B 2503/42

(57) ABSTRACT

Disclosed is a system for evaluating therapeutic effect of mental illness and/or neurodegenerative disease. It is found in the present invention that activation of PV-positive neurons in the primary auditory cortex can generate an effect of preventing depression in advance; when resisting external continuous or intermittent but long-term adverse stimulation, the organism resists the depressive-like behavior that may be caused by increasing the activity of PV-positive neuronal cells in the primary auditory cortex. Therefore, drugs that specifically activate PV-positive neurons in the primary auditory cortex can effectively treat depression, providing new ideas and directions for the development of drugs for depression.

### TECHNICAL FIELD

The present invention relates to the technical field of mental illness, and in particular, to a system for evaluating therapeutic effect of mental illness and/or neurodegenerative disease.

### BACKGROUND OF RELATED ART

At present, a large number of people around the world are troubled by mental illness, which has caused a serious social burden. Moreover, with the rapid development of society, the social burden caused by mental illness continues to grow rapidly. Among mental illnesses, depression is a common mood disorder with significant and persistent low mood as the main clinical feature, and is the main type of mood disorder.

Major depressive disorder has become a public health problem with its high incidence, high disability rate and serious suicidal tendency. With the rapid development of society, the increase of stress factors such as fast pace of life and high competitive pressure has led to the increasing incidence of depression year by year. Among all the social burdens caused by mental illnesses, depression ranks first in both developed and developing countries. Depression has a high disability rate and will become the leading cause of disability. Anhedonia is the main symptom of depression, which is usually accompanied by difficulty concentrating, abnormal appetite, sleep disorders, a sense of worthlessness and even recurrent suicidal tendencies (the vast majority of suicides are caused by depression, which is the leading cause of death among minors), which brings endless pain to patients and their families and seriously endangers human life and health.

Stress is the main cause of depression, and stress can severely disrupt an individual's physiological and psychological balance. Stress-related psychopathology, such as major depressive disorder, anxiety, conduct disorder, and post-traumatic stress disorder, can disrupt individual behavior, cognition, and social interaction and exacerbates people's responses to stressful events. However, traumatic stress does not generate the similar influence on everyone. While susceptible individuals are less adaptable to stress and express inappropriate responses that may become a persistent state of stress, however the individuals with depressive insensitivity can perceive adversity as minimal threat and develop adaptive physiological and psychological responses. In the past decade, the phenomenon of depression non-susceptibility has received increasing attention: most people can maintain normal psychological and physical function and avoid serious mental illness even when exposed to great stress and trauma. In this context, resilience refers to the ability of individuals to avoid the negative social, psychological, and physiological consequences caused by extreme stress that would otherwise damage their psychological or physical health. Recent reports suggest that human adaptability represents a positive, adaptive process, not just the absence of pathological responses in more susceptible individuals.

Regarding the treatment of depression, since the pathogenesis of depression is still unclear, at this stage, the clinical treatment of depression is still mainly based on drug therapy. However, the use of antidepressants faces problems such as slow onset of action, poor medication compliance, low drug efficacy, serious side effects and high relapse rate after stopping medication. Therefore, finding a new antidepressant method with fast onset is a key scientific issue in current depression research.

In the past, research on mental illness and emotional disorders has mainly focused on the prefrontal cortex, hippocampus, amygdala, etc., in the brain, which are believed to be important parts for regulating memory, fear, excitement, anxiety, etc. Abnormalities in these brain regions are closely related to mental illnesses such as anxiety and depression. However, no one has found that the sensory cortex is involved in emotion regulation, especially the primary sensory cortex. It is generally considered that the primary auditory cortex (A1) is mainly involved in the processing of auditory information, and there is no specific research showing that neurons in the auditory cortex may be involved in the processing of emotional information.

Neurons in the brain, which are also called neuronal cells and are the most basic structural and functional units of the nervous system. According to the types of neurotransmitters they release, they are mainly divided into two types: glutamatergic excitatory neurons that release excitatory neurotransmitters such as glutamate and GABAergic inhibitory interneurons that release inhibitory neurotransmitters γ-aminobutyric acid (GABA). CaMKII (calcium/calmodulin-dependent protein kinase II) is specifically expressed in excitatory neurons, so CaMKII-positive neurons are excitatory neurons.

The GABAergic inhibitory interneurons can be further subdivided into parvlbumin-positive neurons (PV-positive neurons), somatostatin-expressing neurons (SST-positive neurons) and other neurons according to the characteristic proteins they express.

In the primary auditory cortex, the PV-positive neurons and SST-positive neurons totally account for about 65% of the inhibitory neurons. Previously, no studies have shown that any of the above neurons in the auditory cortex are involved in neurological diseases such as depression and anxiety. And, it is generally believed that the important manifestations of depression and other diseases are significant and persistent low mood and loss of interest. Therefore, commonly used drugs include tricyclic imipramine, norepinephrine reuptake inhibitor oxadiazine, etc., which can improve neuronal excitability and increase activity of the excitatory neurons.

The Cre/loxP recombinase system is a widely used in vivo gene targeting technology that can achieve conditional, inducible, and spatiotemporal specific gene targeting. Inducible Cre recombinase (cyclization recombinase estrogen receptor; Tamoxifen, Cre-ERT) is a fusion protein containing ligand binding region mutant (ERT) of estrogen receptor (ER) and the Cre recombinase. After intraperitoneal injection of tamoxifen, the metabolite 4-OHT (estrogen analog) of tamoxifen binds to ERT, which allows Cre-ERT2 to enter the nucleus and exert Cre recombinase activity. The FLP/FRT recombinase system is similar to the Cre/loxP recombinase system, wherein FLP is a recombinase (flippase, Flp), and its recognition site is FRT (flippase recognition target, FRT).

### SUMMARY OF THE INVENTION

An object of the present invention is to overcome the above-mentioned deficiencies of the prior art and provide use of an activator of PV-positive neuronal cells in the primary auditory cortex in preparing an antidepressant drug.

A first object of the present invention is to provide use of a product for detecting the activation condition of PV-positive neuronal cells in the primary auditory cortex in preparing a product for diagnosing mental illness and/or neurodegenerative disease, evaluating the risk of mental illness and/or neurodegenerative disease, and/or evaluating the therapeutic effect of mental illness and/or neurodegenerative disease.

A second object of the present invention is to provide a system for evaluating the therapeutic effect of mental illness and/or neurodegenerative disease.

A third object of the present invention is to provide use of a product for activating or enhancing activation of PV-positive neuronal cells in the primary auditory cortex in preparing a product for resisting mental illness and/or neurodegenerative disease.

A fourth object of the present invention is to provide use of a product for activating or enhancing activation of medial geniculate nucleus (MG) neurons projecting to the primary auditory cortex in preparing a product for resisting mental illness and/or neurodegenerative disease.

A fifth object of the present invention is to provide use of a product for activating or enhancing activation of projections from the medial geniculate nucleus to the primary auditory cortex in preparing a product for resisting mental illness and/or neurodegenerative disease.

A sixth object of the present invention is to provide a tangible computer-readable medium encoded with a program.

A seventh object of the present invention is to provide a method for resisting mental illness and/or neurodegenerative disease.

In order to achieve the above objects, the present invention is implemented by the following solutions:

Use of a product for detecting the activation condition of PV-positive neuronal cells in the primary auditory cortex in preparing a product for diagnosing mental illness and/or neurodegenerative disease, evaluating the risk of mental illness and/or neurodegenerative disease, and/or evaluating the therapeutic effect of mental illness and/or neurodegenerative disease.

Preferably, the PV-positive neuronal cells in the primary auditory cortex are PV-positive neuronal cells that specifically activate only the MG projection part of the primary auditory cortex.

Preferably, the mental illness and/or neurodegenerative disease include, but not limited to, one or more of depression, major depression, schizophrenia, bipolar disorder, post-traumatic stress disorder, anorexia, anxiety, dementia (including Alzheimer's disease), drug addiction, deficit and hyperactivity disorder, and autism, etc.

As a specific embodiment, the mental illness and/or neurodegenerative disease is depression.

A system for evaluating therapeutic effect of mental illness and/or neurodegenerative disease includes an information acquisition module, a data processing module and a result output module, where:
the information acquisition module is configured to obtain the activation condition of PV-positive neuronal cells in the primary auditory cortex of a patient before and after treatment, or to obtain the activation condition of PV-positive neuronal cells in the primary auditory cortex of the treatment group and the control group;
the data processing module is configured to compare an activation degree of PV-positive neuronal cells in the primary auditory cortex before and after treatment, or to compare the activation degree of PV-positive neuronal cells in the primary auditory cortex of the treatment group and the control group;
the result output module is configured to output the therapeutic effect:
   if the activation degree of PV-positive neuronal cells in the primary auditory cortex after treatment is higher than that of PV-positive neuronal cells in the primary auditory cortex before treatment, the treatment is effective; otherwise, the treatment is ineffective;
   or, if the activation degree of PV-positive neuronal cells in the primary auditory cortex of the treatment group is higher than that of PV-positive neuronal cells in the primary auditory cortex of the control group, the treatment is effective; otherwise, the treatment is ineffective.

Use of a product for activating or enhancing activation of PV-positive neuronal cells in the primary auditory cortex in preparing a product for resisting mental illness and/or neurodegenerative disease.

Preferably, the PV-positive neuronal cells in the primary auditory cortex are PV-positive neuronal cells that specifically activate only the MG projection part of the primary auditory cortex.

Preferably, the activator specifically activates the PV-positive neuronal cells in the primary auditory cortex.

Use of a product for activating or enhancing activation of medial geniculate nucleus (MG) neurons projecting to the primary auditory cortex in preparing a product for resisting mental illness and/or neurodegenerative disease.

Preferably, the product for activating or enhancing activation of medial geniculate nucleus (MG) neurons projecting to the primary auditory cortex continuously or intermittently but chronically activates the MG neurons projecting to the primary auditory cortex.

Preferably, the medial geniculate nucleus neurons projecting to the primary auditory cortex specifically activate only PV-positive neurons in the primary auditory cortex.

Use of a product for activating or enhancing activation of projections from the medial geniculate nucleus to the primary auditory cortex in preparing a product for resisting mental illness and/or neurodegenerative disease.

Preferably, the mental illness and/or neurodegenerative disease include, but not limited to, one or more of depression, major depression, schizophrenia, bipolar disorder, post-traumatic stress disorder, anorexia, anxiety, dementia (including Alzheimer's disease), drug addiction, deficit and hyperactivity disorder, and autism, etc.

As a specific embodiment, the mental illness and/or neurodegenerative disease is depression.

Preferably, the projection of the medial geniculate nucleus to the primary auditory cortex is the projection of the medial geniculate nucleus to the PV-positive neurons of the primary auditory cortex.

More preferably, resisting mental illness and/or neurodegenerative disease is the treatment and/or prevention of mental illness and/or neurodegenerative disease.

More preferably, the device is one or more of a transcranial magnetic stimulator, an electroencephalogram recorder, a vibrator, a sound frequency generator, and an electric current stimulator.

A tangible computer-readable medium encoded with a program which, when executed, causes a device to perform a treatment method, and the treatment method includes at least one of the following methods:
activating or enhancing the activation of PV-positive neuronal cells in the primary auditory cortex;
activating or enhancing the activation of medial geniculate nucleus neurons projecting to the primary auditory cortex;
activating or enhancing the activation of MG projections to the primary auditory cortex; and
taking less than 14 days of medial geniculate nucleus neuron activity inhibition treatment as a course of treatment;
treating a subject's mental illness and/or neurodegenerative disease or reducing the subject's possibility of developing a mental illness and/or neurodegenerative disease.

Preferably, the treatment includes one or more courses of treatment.

As a specific embodiment, the inhibition of the activity of the medial geniculate nucleus neurons for less than 14 days is: 8 times a day, 10 seconds for each time, light inhibition the activity of the medial geniculate nucleus neurons for three days.

Preferably, the device is an instrument that stimulates neurons through nuclear magnetic resonance, sound waves, vibration or electric current.

A method for evaluating therapeutic effect of antidepressant treatment, including: establishing a social defeat model on experimental animals, randomly dividing the animals into a control group and a treatment group after successful establishment of the model, and carrying out antidepressant treatment on the experimental animals in the treatment group, after treatment, comparing an activation degree of PV-positive neuronal cells in the primary auditory cortex of the experimental animals in the control group and the treatment group. If the activation degree of PV-positive neuron cells in the primary auditory cortex of the treatment group is higher than that of the PV-positive neuron cells in the primary auditory cortex of the control group, the treatment is effective; otherwise, the treatment is ineffective.

A method for treating and/or preventing depression is also claimed in the present invention, and the treatment is carried out by one or more of the following methods:
activating or enhancing the activation of PV-positive neuronal cells in the primary auditory cortex;
activating or enhancing the activation of medial geniculate nucleus neurons projecting to the primary auditory cortex;
activating or enhancing the activation of projections of medial geniculate nucleus to the primary auditory cortex; and
inhibiting the activity of medial geniculate nucleus neurons, taking less than 14 days of activity inhibition treatment as a course of treatment, but not limited to one treatment course.

Preferably, the activation or enhanced activation is performed by using one or more of a transcranial magnetic stimulator, an electroencephalogram recorder, a vibrator, a sound frequency generator, and an electric current stimulator.

Compared with the prior art, the present invention has the following beneficial effects:

It is found in the present invention that early activation of PV-positive neurons in the primary auditory cortex can generate an effect of preventing depression in advance; when resisting external continuous or intermittent but long-term adverse stimulation, the organism resists the depressive-like behavior that may be caused by long-term stimulation through increasing the activity of PV-positive neuronal cells in the primary auditory cortex. Therefore, drugs that specifically activate PV-positive neurons in the primary auditory cortex can effectively treat depression, providing new ideas and directions for the development of depression drugs.

### BRIEF DESCRIPTION OF THE DRAWINGS

FIG. 1 shows that PV-positive neurons in the primary auditory cortex of depression-non-susceptible mice are activated in the social defeat model: a is the co-staining result of CamkII-positive neurons in the primary auditory cortex and c-fos protein in the social defeat model; b is the co-staining result of SST-positive neurons in the primary auditory cortex and c-fos protein in the social defeat model; c is the co-staining result of PV-positive neurons in the primary auditory cortex and c-fos protein in the social defeat model; d is the three states of mice in the social defeat model; e is the calcium signal result of PV-positive neurons in the primary auditory cortex of RES mice (depression-non-susceptible mice); f is the calcium signal result of PV-positive neurons in the primary auditory cortex of SS mice (depression-susceptible mice).

FIG. 2 shows that compound-directed activation of PV-positive neurons in the primary auditory cortex can generate antidepressant-like effects: a shows that compound-directed activation of PV-positive neurons in the primary auditory cortex verifies its antidepressant effect through FST experiment; b shows that compound-directed activation of PV-positive neurons in the primary auditory cortex does not affect the motor ability of mice; c shows that compound-directed activation of PV-positive neurons in the primary auditory cortex can reverse depressive-like behavior.

FIG. 3 shows that the chemical genetic method specific activation of PV-positive neurons in the primary auditory cortex can produce antidepressant-like effects: a shows that the chemical genetic method specific activation of PV-positive neurons in the primary auditory cortex verifies its antidepressant effect through FST experiments; b shows that the chemical genetic method specific activation of PV-positive neurons in the primary auditory cortex does not affect the motor ability of mice; c shows that the chemical genetic method specific activation of PV-positive neurons in the primary auditory cortex can significantly reduce the possibility of depressive-like behavior caused by stress; d shows that the chemical genetic method specific activation of PV-positive neurons in the primary auditory cortex can reverse depressive-like behavior.

FIG. 4 shows that the PV-positive neurons in the primary auditory cortex are mainly regulated by neurons in the MG brain region: a is the main brain region of PV-positive neurons projecting to the primary auditory cortex; b is the MG neuron type of PV-positive neurons projecting to the primary auditory cortex.

FIG. 5 shows that specific activation of MG brain region neurons projecting to the primary auditory cortex can generate antidepressant-like effects: a shows that the specific activation of MG brain region neurons projecting to the primary auditory cortex verifies its antidepressant effect through FST experiments; b shows that the specific activation of MG brain region neurons projecting to the primary auditory cortex does not affect the motor ability of mice; c shows that the specific activation of MG brain region neurons projecting to the primary auditory cortex can significantly reduce the possibility of depressive-like behavior caused by stress; d shows that the specific activation of MG brain region neurons projecting to the primary auditory cortex can reverse depressive-like behavior.

FIG. 6 shows that specific activation of PV-positive neurons in the primary auditory cortex receiving projections from the MG brain region can produce antidepressant-like effects: a shows that specific activation of PV-positive neurons in the primary auditory cortex receiving projections from the MG brain region verifies its antidepressant effect through FST experiments; b shows that specific activation of PV-positive neurons in the primary auditory cortex receiving projections from the MG brain region does not affect the motor ability of mice; c shows that specific activation of PV-positive neurons in the primary auditory cortex receiving projections from the MG brain region can reverse depressive-like behavior.

FIG. 7 shows that anti-depressant individuals appears reduced activity of MG brain region neurons projecting to the primary auditory cortex in the early stage of stress stimulation: a is the calcium activity of MG brain region neurons projecting to the primary auditory cortex of SS mice (depression-susceptible mice) in the early stage of stress stimulation; b is the calcium activity of MG brain region neurons projecting to the primary auditory cortex of RES mice (depression-non-susceptible mice) in the early stage of stress stimulation; c shows that the calcium signal activity of mice when they were attacked in the early stage (day 2) is negatively correlated with the Interaction Ratio value after the end of the 10-days modeling.

FIG. 8 shows that short-term inhibition of MG brain region neurons projecting to the primary auditory cortex can activate PV-positive neuronal cells in the primary auditory cortex, thereby having an antidepressant effect: a shows that short-term inhibition of MG brain region neurons projecting to the primary auditory cortex can activate PV-positive neuronal cells in the primary auditory cortex; b shows that short-term inhibition of MG brain region neurons projecting to the primary auditory cortex can significantly reverse the depressive-like phenotype.

### DETAILED DESCRIPTION OF THE EMBODIMENTS

The present invention is further described in detail below in conjunction with the drawings and specific embodiments of the specification. The embodiments are only used to explain the present invention and are not used to limit the scope of the present invention. The experiment methods used in the following embodiments are conventional methods unless otherwise specified; the materials, reagents, etc., are commercially available reagents and materials unless otherwise specified.
1. Immunofluorescence labeling antibodies

Primary antibodies: mouse anti-PV (Swant company, Article No.: 235), mouse anti-CamkIIα (Invitrogen company, Article No.: MA1-048), rabbit anti-c-fos (CST company, Article No.: 2250s)
2. Experimental animals

Male C57BL/6J mice (8-12 weeks old) used in the experiment are from the Experimental Animal Center of Southern Medical University (License No.: SCXK-2011-0015, Guangzhou, China).

The sources of the transgenic mouse strains used are as follows:

PV-Cre (No.: 008069), SST-Cre (No.: 013044), CamkII-Cre^{ERT} (No.: 012362) and ROSA26-tdTomato reporter mice (No.: 007909) were purchased from The Jackson Laboratory, USA.

PV; tdTomato mice were obtained by mating PV-Cre and ROSA26-tdTomato, SST; tdTomato mice were obtained by mating SST-Cre and ROSA26-tdTomato, and CamkII-Cre^{ERT}; tdTomato mice were obtained by mating CamkII-Cre^{ERT} and ROSA26-tdTomato.

All transgenic mouse strains are on C57BL/6J background.
3. Viruses and article numbers used: The following viruses are from Wuhan Shumi Technology Co., Ltd.: rAAV-Ef1a-DIO-mCherry-WPRE-pA (Article No. PT-0013), rAAV-Ef1a-DIO-hM3D(Gq)-mCherry-WPRE-pA (Article No. PT-0042),
rAAV-Ef1a-DIO-GCaMp6f-WPRE-hGH pA (Article No. PT-0106-9),
rAAV-hSyn-CRE-WPRE-hGH polyA (Article No. PT-0136),
RV-ENVA-ΔG-dsRed (Article No. R01002),
rAAV-Ef1a-DIO-His-EGFP-2a-TVA-WPRE-pA (Article No. 9-21-K180918),
rAAV-CMV-DIO-RVG-WPRE-pA (Article No. 9-991-K180726),
rAAV-nEF1a-fDIO-eNpHR3.0-mCherry-WPRE-hGH polyA (Article No. PT-1261),
rAAV2/R-hSyn-NLS-FLP-bGH pA (Article No. PT-0133).

The following viruses are from Shanghai Taiertu Biotechnology Co., Ltd.:
AAV2/1-CAG-FLEX-Flpo-WPRE-pA (Article No. S0273-9),
AAV2/9-hEF1a-fDIO-mCherry-WPRE-pA (Article No. S0553-9),
AAV2/9-hEF1a-fDIO-hM4D(Gi)-mCherry-ER2-WPRE-pA (Article No. S0336-9),
AAV2/9-hEF1a-fDIO-hM3D(Gq)-mCherry-ER2-WPRE-pA (Article No. S0335-9),
AAV2/2Retro-CAG-FLEX-Flpo-WPRE-pA (Article No. S0273-2/R).
4. Animal feeding conditions

The experimental mice were all raised in exhaust ventilated closed cages (EVC), 4-5 mice/cage, and the feeding environment followed the standard laboratory environment, with the temperature controlled at 24 °C, 12-hour day and night cycle (7:00 A.M. to 7:00 P.M.), and free access to food and water. Before the animal behavior test was carried out, all mice were touched for 3-4 days (2 times/day, 5 min/time). The animal behavior test was carried out between 1:00 P.M. and 5:00 P.M. All animal experimental operations strictly followed the relevant regulations of the Chinese Animal Ethics Committee.
5. Gene identification

After the transgenic mouse offspring was born, a small number of tissues (ears, toes, tail) was cut, and DNA was extracted by boiling with DNA extraction solution for 1 hour, and then PCR and agarose gel nucleic acid electrophoresis were used to identify the genes of transgenic mice.
6. Immunofluorescence staining

Brain slice tissue processing and immunohistochemistry methods were performed in accordance with the methods used in this laboratory before. After deep anesthesia, mice were first perfused with 0.1M PBS and 4% formaldehyde solution. After perfusion, the mouse brain was dissected out and fixed in 4% formaldehyde solution for 4 to 6 hours, then rinsed with tap water for 1 hour, and finally immersed in 30% saturated sucrose solution. After the mouse brain was precipitated with sucrose, it was sliced using the Leica freezing microtome (CM1850, Lecia, Nussloch, Germany), and coronal brain slices containing A1 (thickness: 40 μm) were cut. The brain slices were first blocked with goat serum blocking solution containing 0.3% triton X-100 for 2 hours, then incubated with primary antibody overnight (4 °C), then rinsed with 0.1 M PBS (5 min×3) and incubated with secondary antibody for 2 hours at room temperature in the dark. Finally, the brain slices were mounted with mounting medium (Vectro Laboratories Inc.) containing DAPI. Eight A1-containing brain slices from a single mouse brain were selected for carrying out colocalization analysis of c-fos with CamkII, PV, and SST, respectively.

Immunofluorescence imaging was performed using a Nikon A1R confocal microscope (Nikon Instruments Inc, Japan), and positive cell counts were performed using Image J analysis software (US National Institutes of Health).
7. Stereotactic technique (microinjection, cannula implantation)

After anesthesia, the mouse was fixed on a brain stereotaxic instrument, the hair on the top of the head was removed, the skin was disinfected, a midline incision was made to expose bregma, and the mouse brain was positioned according to the mouse brain atlas. For in vivo adenovirus injection, 0.1 μL of virus was separately injected into the bilateral MG of the mouse and 0.15 μl was separately injected into the bilateral A1 of the mouse. The virus was injected at a rate of 0.03 μl min⁻¹ using a 33 GA microinjection needle (Hamilton) and a microinjection pump (Stoelting, Wood Dale, IL). The needle of the microinjection needle was positioned at MG (AP=−2.92 mm; ML=±1.95 mm; DV=3.5 mm), A1 (AP=−2.45 mm; ML=±4.25 mm; DV=0.8 mm), and a cranial drill was used to drill holes above MG/Au1 on both sides. The needle was inserted according to the required depth, and the virus was injected at a rate of 0.03 μl min⁻¹. After the injection, the needle was retained for 5 min, then the needle was slowly withdrawn and finally is sutured. All experiments were performed 3 weeks after the administration of the virus. After the experiment was completed, the virus injection site was confirmed by intraperitoneal injection of CNO (2 mg/kg) and detection of mcherry expression.

When using stereotactic technology for cannula implantation, the cannula was implanted bilaterally in A1 (AP=−2.45 mm; ML=±4.25 mm; DV=0.7 mm). Relevant pharmacological experiments were carried out after the mice recovered for 1 week.
8. Social defeat model

The social defeat model was conducted with reference to existing literature. The social defeat model was conducted using CD1 retired breeder mice with aggressive behavior. The experimental mice were attacked by different CD1 retired breeder mice for 10 minutes every day for 10 consecutive days.

After each attack, the experimental mice and CD1 retired breeder mice were separated by a transparent plexiglass partition plate with air holes, and rotated before being attacked the next day. The control mice were also kept two per cage, separated by a transparent plexiglass partition plate with air holes, and rotated once a day.

Social interaction behavior detection: social interaction behavior was detected 24 hours after the last aggressive behavior (day 11). The detection process includes two 2.5 minutes. In the first 2.5 minutes, the experimental mice were placed in a new test box and allowed to move freely. One side of the test box is placed with a small plexiglass frame with air holes. Then, after 1 min of returning the mouse to the original cage, the mouse was returned to the test box again and the activity of the second 2.5 min was recorded. At this time, a strange CD1 retired species (Target) was placed in the plexiglass frame with air holes. The activity time of the mouse in the interaction area was tracked using Ethovision 7.0 software (Noldus, Wageningen, Netherlands). (Interaction area time, target)/(Interaction area time, no target)×100 was defined as Interaction Ratio.

The depression-susceptible group (SS) and the depression-non-susceptible group (RES) were distinguished using Interaction Ratio. When the Interaction Ratio was less than 100, it is the depression-susceptible group, and when the Interaction Ratio was greater than or equal to 100, it is the depression-non-susceptible group. In the experiments after modeling, the mice were raised in single cages.

All aggressive behaviors were performed between 16:00 and 17:00. Social interaction behavior tests were all conducted between 18:00 and 24:00, and the mice to be tested were placed in the test room 1 hour in advance for dark adaptation.
9. Forced swimming test

The forced swimming test was conducted with reference to existing reports. The mice to be tested were placed in the test room 1 hour in advance to adapt. A cylindrical transparent glass tank (height 45 cm, inner diameter 19 cm) was used for the test, and water (water depth 23 cm, water temperature 22-25 °C) was added. The mice were gently placed in the tank and allowed to swim freely in the tank for 6 minutes. The immobility time of the mice was recorded in the last 4 minutes. The higher the immobility time of the mice, the higher the depression degree of the mice. Conversely, the lower the immobility time, the more anti-depressant the mice are.
10. Open-field test

The mice to be tested were placed in the test room 1 hour in advance to adapt. The test equipment was an open test box (Accuscan Instruments, Columbus, OH) surrounded by transparent plexiglass. The mice were placed in the test box and allowed to explore freely for 5 minutes. The total distance of the movement of mice was analyzed using Versmax analysis software.
11. Data analysis

All data were statistically analyzed using GraphPad Prism statistical software. Statistical analysis methods include Teo-tailed Student's t-tests, One-way ANOVA, and Two-way NOVA. When the P value is less than 0.05, it is considered to have a significant difference. The experimental results were all graphed using GraphPad Prism graphing software.

### Embodiment 1 Number Increase and Activity Significant Enhancement of PVs in Primary Auditory Cortex Region of Depression-Insensitive Mice

### 1. Experimental Method

In order to explore the reason why mice in the depression-non-susceptible group have antidepressant ability in the social defeat model, we used fluorescent reporter mice labeled with different types of neurons (that is, the corresponding neurons can express red fluorescence). After modeling social defeat model, observe whether various neurons in the primary auditory cortex of the mouse brain are activated or inhibited, that is, analyze whether there are certain types of neurons in the primary auditory cortex of the brain are involved in anti-depression.

The specific method is as follows:
Use each-neuron fluorescent reporter mice obtained by hybridization:
   PV; tdTomato mice (PV-positive neuron reporter mice), SST; tdTomato mice (SST-positive neuron reporter mice) and CamkII-Cre^{ERT}; tdTomato mice (red fluorescent reporter mice of excitatory neurons).

The three kinds of fluorescent reporter mice were respectively used to model the classic depression model-social defeat model (CSDS). According to the detection results of social interaction behavior 10 days after modeling, the mice were divided into: (1) normal control group (CTRL): those who did not experience social defeat; (2) depression-susceptible group (SS): those who experienced social defeat and whose social value (Interaction Ratio) was less than 100; (3) depression-non-susceptible group (RES): those who experienced social defeat and whose Interaction Ratio was greater than 100.

Immediate early genes are a group of genes that are rapidly and briefly activated after a series of external stimulation. The earliest discovered immediate early genes in cells include c-fos, c-myc, and c-jun, etc. Immunofluorescence staining is usually used to observe whether c-fos is expressed to determine whether the neuron is activated or in a silent state: the neurons that express c-fos protein indicate that the neuron is rapidly activated after external stimulation, and conversely, neurons without c-fos protein are in a silent state.

The primary auditory cortex of the three kinds of neuron fluorescent reporter mice, which were grouped according to the results of interaction behavior detection after the above modeling, were subjected to c-fos immunofluorescence staining to analyze whether the three kinds of neurons labeled on the three kinds of fluorescent reporter mice appeared to be activated or silenced after the external stimulation of the social defeat modeling.

### 2. Experimental Results

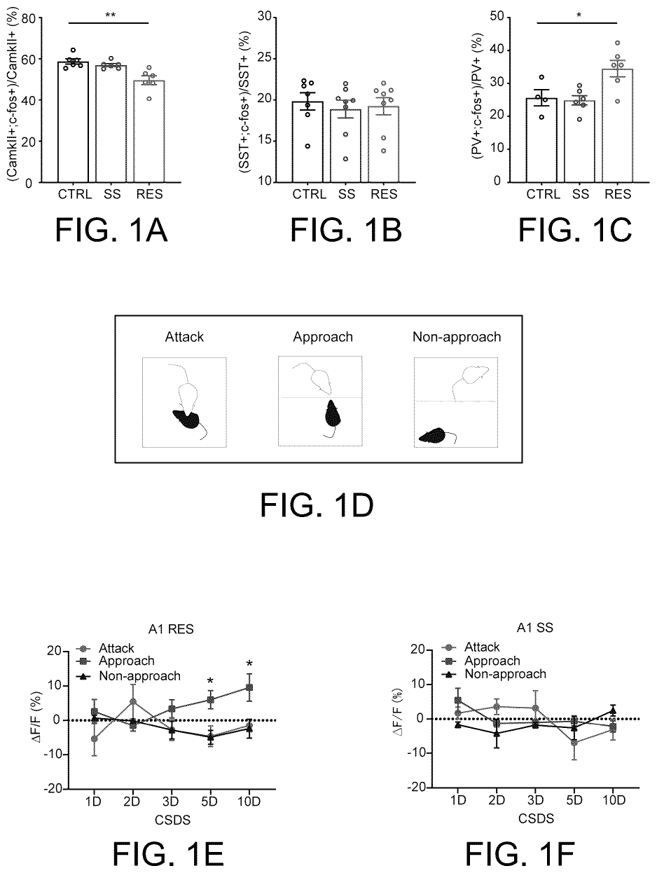

As shown in FIG. 1a, among the excitatory neurons, compared with the normal control group (CTRL) and the depression-susceptible group (SS), in the depression-non-susceptible group (RES), the cell number of CamkII-positive neuron cells also being labeled by c-fos accounted for a significant decrease in the proportion of total CamkII-positive neuronal cells, indicating that the excitatory neurons in the primary auditory cortex of the depression-non-susceptible group (RES) were significantly inhibited.

As shown in FIG. 1b and FIG. 1c, in inhibitory neurons, compared with the normal control group (CTRL), the depression-non-susceptible group (RES) mice showed a significant increase in the number of cells labeled with c-fos in PV-positive neuronal cells, accounting for a significantly higher proportion of the total PV-positive neuronal cells; compared with the normal control group (CTRL), the depression-susceptible group (SS) showed no significant change in the proportion of PV positive neuronal cells that were also labeled with c-fos. However, in another kind of inhibitory neuron, SST-positive neurons, compared to the normal control group (CTRL), the depression-non-susceptible group (RES) mice showed no change in the proportion of the cells co-labeled with SST-positive neurons and c-fos in the primary auditory cortex. The above results indicate that the activity of CamkII positive neurons (excitatory neurons) in FIG. 1a is inhibited by the activation of PV positive neurons.

In summary, in the depression-non-susceptible group of mice established by the classic depression modeling, the activity of PV-positive neurons in the primary auditory cortex was significantly enhanced, and the activation function of PV-positive neuronal cells in this brain region was dominated (compared to CamkII and SST-positive neurons), and it can inhibit the activity of excitatory neurons, showing the key role of activating PV-positive neurons in the primary auditory cortex in antidepressant applications.

In addition, it was found that before the social defeat model was established, the mice in the depression-non-susceptible group had an antidepressant-like phenotype without any treatment, and the activation of PV-positive neurons was a characteristic of the depression-non-susceptible mice, that is, the activation of PV-positive neurons was an instinctive response of antidepressant individuals to resist against depression. Therefore, it can be judged that early activation of PV-positive neurons in the primary auditory cortex can generate an effect of preventing depression in advance.

### Embodiment 2 Real-Time Detection of Changes in PV-Positive Neurons in the Primary Auditory Cortex of Depression-Non-Susceptible Mice

Calcium imaging is a method of detecting calcium ion concentration in tissues by using calcium ion indicators. Calcium imaging is mainly used in nervous system research, where calcium ion changes indicate neuronal activity. Fiber photometry is a commonly used method for recording calcium signals in vivo, which can be used to check and record changes in cell activity in real time.

The calcium imaging method was used to detect the activity condition of PV-positive neurons in the primary auditory cortex of mice in real time while the animal was alive.

### 1. Experimental Method

The Cre enzyme-dependent virus rAAV-Ef1a-DIO-GCaMp6f-WPRE-hGH pA (Article No. PT-0106-9) expressing calcium ion indicator and the optical fiber plug used for optical fiber recording were stereo-injected into the primary auditory cortex of PV-Cre mice. After the virus was injected, it was cleaved by Cre enzyme to express the GCaMP6f calcium indicator protein with green fluorescence. After being excited by the excitation light with a wavelength of 470 nm, its fluorescence signal was collected and recorded by the corresponding program in the computer.

After 14 days, the social defeat model was established for 10 days, and the activity of PV-positive neurons in the primary auditory cortex was recorded at the same time.

After 10 days of modeling, the mice were divided into depression-susceptible group (SS) and depression-non-susceptible group (RES) by social interaction detection.

To analyze the changes in cell activity of PV-positive neurons in the primary auditory cortex of mice in depression-susceptible group (SS) and depression-non-susceptible group (RES) under various modeling conditions, and to analyze in which process these neurons change when experiencing the stress environment of social defeat.

### 2. Experimental Results

During the modeling process of the social defeat model, the modeling mice were mainly divided into three states (FIG. 1d as a schematic diagram): CD1 retired breeder mice attacked C57BL/6J mice (Attack); after separated by plexiglass partition plate with air holes, CD1 retired breeder mice approached (tried to approach) C57BL/6J mice (Approach); the CD1 retired breeder mice left C57BL/6J mice (Non-approach).

As shown in FIG. 1e, the activity of PV-positive neuronal cells in the primary auditory cortex of mice in the depression-non-susceptible group (RES) increased slightly when being attacked in the early stage (2 days). After 5 days of modeling, the approach of the retired breeder mice makes the mice feel threatening stimulation. In response to this approach stimulation, the activity of PV-positive neurons in the primary auditory cortex of the mice increased significantly, and this continued until the end of the last 10 days of modeling.

However, the activity of PV-positive neurons in the primary auditory cortex of mice in the depression-susceptible (SS) group (FIG. 1f) did not change significantly during the modeling process, indicating that during the development of depression, the PV-positive neuronal cells in the primary auditory cortex are not involved.

The above results indicate that when resisting against continuous or intermittent but long-term adverse stimulation from the outside world (during stress response), organisms resist the depressive-like behavior that may be caused by long-term stimulation by increasing the activity of PV-positive neuronal cells in the primary auditory cortex. Therefore, specific activation of PV-positive neurons through nuclear magnetic resonance, sound waves, vibration or electric current can have the effect of treating mental and neurological diseases such as depression.

### Embodiment 3 Activation of PV-Positive Neurons in the Primary Auditory Cortex Can Generate Antidepressant-Like Effects

### 1. Experimental Method

Based on the experimental results of Embodiments 1 and 2, in individuals (RES) with the ability to resist stress stimulation and show antidepressant effects, the activation of PV-positive neurons in the primary auditory cortex plays a key role. Based on this conclusion, a pharmacological method was further used to activate the PV-positive neurons in the primary auditory cortex for behavioral testing.

Muscimol is an activator of inhibitory receptors (GABA) and can widely activate inhibitory neurons. However, some studies have found that muscimol has selectivity for PV-positive neurons among inhibitory neurons. Therefore, muscimol can cause enhanced activity of PV-positive neurons.

(1) The commonly used experimental models for rapid detection of antidepressant effects: forced swimming test (FST) and open field test (OFT) were used to study whether pharmacological activation of PV-positive neurons in the primary auditory cortex can show antidepressant effects.

Specifically: C57BL/6J mice were cannulated in the bilateral primary auditory cortex. Three days later, 1 μl of 10 mM muscimol was administered directly to the primary auditory cortex through the cannula (muscimol as medication group), and the control group was given the same dose of normal saline (saline as control group). After 10 min, a forced swimming test (FST) was performed. Three days later, the drug was administered again (1 μl of 10 mM muscimol was administered to the muscimol medication group, and 1 μl of normal saline was administered to the saline control group), and an open field test (OFT) was performed 10 min later.

Further, C57BL/6J mice were used to establish a 10-day social defeat model to study the antidepressant effect of pharmacological activation of PV-positive neurons in the primary auditory cortex.

Specifically: After 10 days of social defeat model (CSDS) establishment, social interaction behavior was tested to obtain depression-susceptible group mice. Cannulas were implanted in the bilateral primary auditory cortex of the depression-susceptible mice. Three days later, the mice were randomly divided into two groups and given 1 μl of 10 mM muscimol (muscimol medication group after modeling) and the same dose of normal saline (saline control group after modeling), and social interaction behavior was tested again 10 minutes later.

### 2. Experimental Results

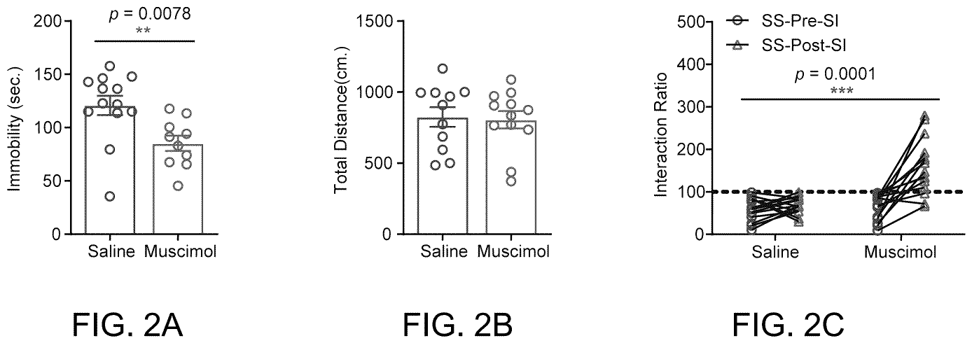

The results are shown in FIG. 2a. In the FST experiment, compared with the saline control group, the muscimol medication group activated PV-positive neurons in the primary auditory cortex. When tested ten minutes after administration, the immobility time was significantly reduced, indicating that the mice in the muscimol medication group had obvious antidepressant-like effects, indicating that drug site-specific activation of GABAergic inhibitory interneurons (PV-positive neurons) in the primary auditory cortex has a rapid antidepressant effect.

The results shown in FIG. 2b show that in the OFT experiment, compared with the saline control group, the total distance of the movement of mice in the muscimol medication group did not change, indicating that muscimol did not affect the motor ability of mice in spontaneous activities. Once again it showed that the reduction in immobility time of the drug-treated mice in the FST was a significant antidepressant phenotype rather than a change in motor ability. That is, the muscimol medication group had significant antidepressant performance compared with the saline control group.

The results shown in FIG. 2c show that, after the social defeat model was established, the Interaction Ratio was significantly increased after the primary auditory cortex of the depression-susceptible group mice was treated with muscimol, while the mice given normal saline had no behavioral phenotype changes. This shows that after modeling, mice can maintain a depressed state, and drug activation of PV-positive neurons in the primary auditory cortex can significantly relieve the depressive-like behavior of mice, indicating that drug activation of PV-positive neurons in the primary auditory cortex has significant therapeutic effects on depressive-like behavior.

In summary, the use of GABA receptor agonist (muscimol) for site-specific activation of inhibitory neurotransmitter GABA receptors (including PV-positive neuronal cells) in the primary auditory cortex can induce rapid antidepressant effects in mice; in addition, the site-specific activation of GABA receptors (including PV-positive neuronal cells) in the primary auditory cortex by GABA receptor agonists can also reverse the behavioral phenotypes of depression-susceptible group mice. It is suggested that depression can be rapidly treated by activating GABA neurons (including PV-positive neurons) in the primary auditory cortex using drugs such as GABA agonists.

### Embodiment 4 Specific Activation of PV-Positive Neurons in the Primary Auditory Cortex by Chemical Genetic Technology Can Generate Antidepressant-Like Effects

Chemical genetic technology (or pharmacogenetics technology) is designer receptors exclusively activated by designer drugs (DREADDs) technology, which changes the structure of the G protein-coupled receptor-acetylcholine receptor, so that it can only be activated or inhibited by a specific compound clozapine-N-oxide (CNO). The modified hM3Dq depolarizes neurons after inducing by CNO, promotes the discharge activity of neurons, and enhances the excitability of neurons.

### I. Specific Activation of PV-Positive Neurons in the Primary Auditory Cortex Can Generate Antidepressant-Like Effects

By using Chemical genetics method to specifically activate PV-positive neuronal cells in the primary auditory cortex, we explored whether this method can generate antidepressant effects.

### 1. Experimental Method

PV-cre transgenic mice were randomly divided into two groups. The primary auditory cortex of mice in the two groups were separately microinjected with chemical genetic virus rAAV-Ef1a-DIO-hM3D(Gq)-mCherry-WPRE-pA (Article No. PT-0042) (hM3Dq group) expressing the carried modified hM3Dq gene in the infected cells, and with the control virus rAAV-Ef1a-DIO-mCherry-WPRE-pA (Article No. PT-0013) (mCherry group) expressing only red fluorescent protein after infecting cells. After 21 days, 2 mg/kg CNO was intraperitoneally injected, and the forced swimming test (FST) was performed 35 minutes later. After 3 days, 2 mg/kg CNO was intraperitoneally injected, and the open field test (OFT) was performed 35 minutes later. The effect of this method on the antidepressant phenotype of mice was determined by behavioral testing.

### 2. Experimental Results

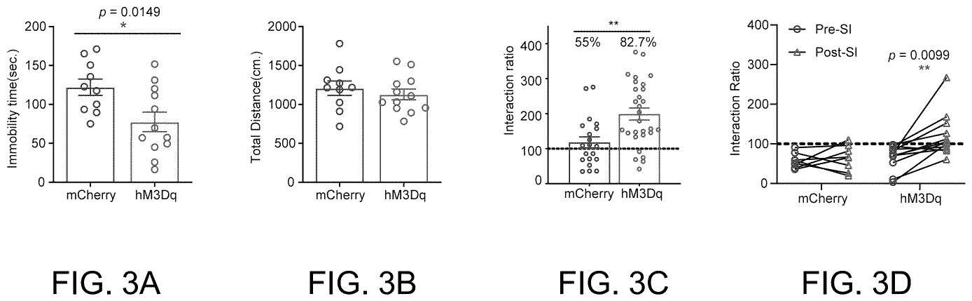

The results in FIG. 3a show that after CNO injection in the hM3Dq group, PV-positive neuronal cells were activated, which significantly reduced the immobility time of mice in forced swimming; while the results in FIG. 3b shows that the total distance of the movement of mice showed that the motor ability of mice was not affected and the spontaneous activity was not affected. This shows that specific activation of PV-positive neurons in the primary auditory cortex can generate antidepressant-like effects.

### II. The Effect of Specific Activation of PV-Positive Neurons in the Primary Auditory Cortex on Preventing Depression

### 1. Experimental Method

The primary auditory cortex of two randomly divided groups of PV-cre mice were separately injected with the chemical genetic virus rAAV-Ef1a-DIO-hM3D(Gq)-mCherry-WPRE-pA (Article No. PT-0042) (hM3Dq group) and the control virus rAAV-Ef1a-DIO-mCherry-WPRE-pA (Article No. PT-0013) (mcherry group).

The social defeat model was established for 10 days. During the 10-day modeling process, 2 mg/kg CNO was intraperitoneally injected twice a day in the morning and evening to keep the PV-positive neurons in the primary auditory cortex in an activated state. After 10 days of modeling, social interaction behavior tests were performed. The proportion of depression-non-susceptible mice in the hM3Dq group injected with chemical genetic virus and the mcherry group injected with control virus after the same modeling was compared.

### 2. Experimental Results

The results in FIG. 3c show that the social interaction test after the social defeat model was established showed that the proportion of non-susceptible groups (Interaction Ratio greater than 100) in the mcherry group injected with control virus was 55%, while the proportion of non-susceptible groups in the hM3Dq group injected with chemical genetic virus was 82.7%, showing a significant upward trend, and the total Interaction Ratio of the hM3Dq group was significantly higher than that of the mcherry group.

The above results show that when receiving adverse stimulation and maintaining in a stressful environment, continuous activation of PV-positive neurons in the primary auditory cortex can significantly increase the proportion of mice in the depression-non-susceptible group, that is, reduce the occurrence of depression caused by stress and prevent the occurrence of depression after stress. Activating PV-positive neurons in the primary auditory cortex can resist the occurrence of depression caused by long-term and continuous stress stimulation.

### III. Therapeutic Effect of Activating PV-Positive Neurons in the Primary Auditory Cortex After Depression Appearance

### 1. Experimental Method

The primary auditory cortex of two randomly divided groups of PV-cre mice were separately injected with the chemical genetic virus rAAV-Ef1a-DIO-hM3D(Gq)-mCherry-WPRE-pA (Article No. PT-0042) (hM3Dq group) and the control virus rAAV-Ef1a-DIO-mCherry-WPRE-pA (Article No. PT-0013) (mcherry group).

After 21 days, the social defeat model was established to obtain depression-susceptible mice with an Interaction Ratio of less than 100. Among them, the mice were divided into the depression-susceptible hM3Dq group and the depression-susceptible mcherry group according to the virus injected 21 days ago. All mice were given intraperitoneal injection of CNO to activate the PV-positive neurons in the primary auditory cortex of the depressed mice in the hM3Dq group. The social interaction behavior was detected 35 minutes after the intraperitoneal injection, and the depressive-like behavior of the mice was observed.

### 2. Experimental Results

The results in FIG. 3d show that Pre-SI is the Interaction Ratio measured by the first test after modeling to separate the depressed mice (Interaction Ratio less than 100), and Post-SI is the Interaction Ratio measured 35 minutes after CNO administration.

The mcherry group injected with the control virus did not improve the depressive phenotype of mice after CNO administration, indicating that the mice will maintain depressive-like behavior after modeling, and the injection of the virus itself has no effect, and CNO itself has no antidepressant effect. While, after the chemical genetic virus-injected hM3Dq group was injected with CNO to activate the PV-positive neurons in the primary auditory cortex, the Interaction Ratio of SS mice in Pre-SI increased (Post-SI after CNO injection), and the depressive-like phenotype was significantly improved, indicating that the specific activation of PV-positive neuronal cells in the primary auditory cortex can significantly reverse the depressive phenotype and make it present an anti-depressive phenotype.

In summary, in the case of long-term or frequent external stress stimulation in life, the specific activation of PV-positive neurons in the primary auditory cortex through chemical genetics can prevent and reduce the occurrence of depressive-like behavior after stress stimulation, and greatly reduce the occurrence of depression; if the PV-positive neurons in the primary auditory cortex are activated after depression occurs, it can also relieve and treat depressive symptoms. This provides a new way for clinical disease treatment and finds an effective means to prevent the occurrence of mental and neurological diseases.

### Embodiment 5 PV-Positive Neurons in the Primary Auditory Cortex Are Mainly Regulated by Neurons in the MG Brain Region

Retrograde trans-monosynaptic rabies virus (Rhabdoviridae, RV) neural circuit tracing method. After RV infects the central nervous system, it mainly labels neurons and almost does not label glial cells. The replication-deficient recombinant RV constructed based on the infectious clone of the RV vaccine strain Sad-B19 has low toxicity and high safety, can clearly label the fine morphology of neurons, and achieves reverse trans-monosynaptic tracing of neural network connections through the reverse complementation strategy. Using Cre transgenic mice combined with Cre-LoxP to control the expression of TVA and G protein in AAV helper virus, it is possible to realize the expression of TVA and G protein only in specific kinds of neurons in specific regions, thereby using RV-EnvA-ΔG to achieve reverse trans-monosynaptic labeling of specific kinds of neurons.

Clarifying how PV-positive neurons in the primary auditory cortex are regulated can increase the pathways and means of activating PV-positive neurons in A1.

### I. PV-Positive Neurons in the Primary Auditory Cortex Are Mainly Regulated by Neurons in the MG Brain Region

### 1. Experimental Method

By using the neural circuit tracing method of retrograde trans-monosynaptic rabies virus (Rhabdoviridae, RV), the brain region and neurons that control PV in the primary auditory cortex were found.

Specifically, on day 1, the primary auditory cortex of PV-cre mice were stereotactically microinjected with cre expression-dependent AAV virus rAAV-Ef1a-DIO-His-EGFP-2a-TVA-WPRE-pA (Article No. 9-21-K180918) for specific infection of ENVA-encapsulated viruses for expressing TVA, and AAV virus rAAV-CMV-DIO-RVG-WPRE-pA (Article No. 9-991-K180726) for assisting rabies virus retrograde trans-monosynaptic expression of RVG.

On the 15th day, the modified pseudorabies virus RV-ENVA-AG-dsRed (Article No. R01002) was injected into the same position. It recognizes TVA and retrogradely transports it to the upper level neurons via RVG axons to express the red fluorescent protein reporter gene it carried.

On the 21st day, perfusion, post-fixation, sugar precipitation and whole brain cryosection were performed.

By analyzing the brain region where the red fluorescence is located, it can be known which brain region the PV-positive neurons in the primary auditory cortex receive projections from.

### 2. Experimental Results

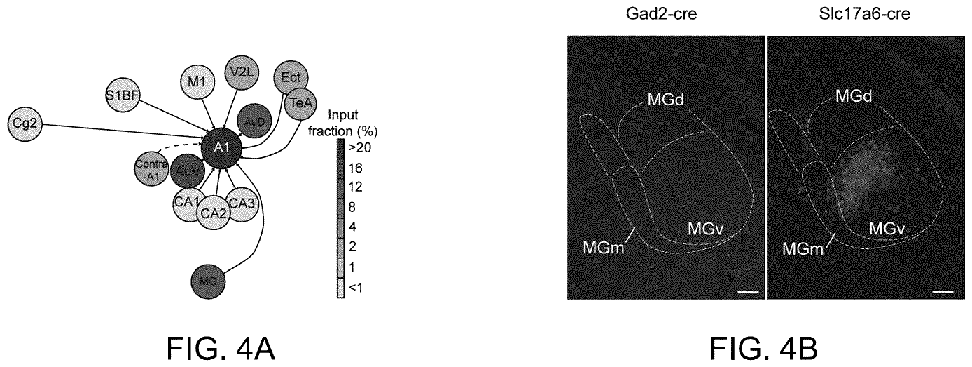

As shown in FIG. 4a, the brain regions of PV-positive neurons projecting to the primary auditory cortex are mainly the primary auditory cortex local brain region (A1) (41.17%), and the surrounding secondary auditory cortex, including: ventral secondary auditory cortex (AuV) (16.69%), dorsal secondary auditory cortex (AuD) (15.86%). In addition, among other brain regions, the medial geniculate nucleus (MG) is the main projection brain region, accounting for 16.58%, and the rest totals 9.7%.

It is found that in addition to the internal projection of the auditory cortex, most of the external projections of PV-positive neuronal cells projecting to the primary auditory cortex come from MG.

Therefore, by enhancing the projection of MG to the primary auditory cortex or enhancing the projection of MG to PV-positive neurons in the primary auditory cortex, the excitability and activity of PV-positive neurons can also be enhanced, thereby achieving the purpose of anti-depression or treatment of depression.

### II. Regulation of the MG Brain Region Neurons in the Primary Auditory Cortex Into Excitatory Neurons

### 1. Experimental Method

The characteristic protein of excitatory glutamatergic neurons, the glutamate transporter protein 2 gene (Vglut2, also known as Slc17a6), is expressed in multiple brain regions. Slc17a6-cre mouse was used to label excitatory neurons, and Gad2-cre mouse was used to label GABAergic inhibitory interneurons. The retrogradely projecting AAV2/2Retro-CAG-FLEX-Flpo-WPRE-pA (Article No. S0273-2/R) was injected into the primary auditory cortex of the two cre mice, and AAV2/9-hEF1a-fDIO-mCherry-WPRE-pA (Article No. S0553-9) was injected into the MG at the same time. After 21 days, the mice were perfused and sliced to observe the red fluorescent labeling of the MG.

### 2. Experimental Results

As shown in FIG. 4b, there is no red fluorescence in the MG brain region of Gad2-cre mouse, indicating that the neurons projecting to the primary auditory cortex are not inhibitory neurons; while the MG brain region of Slc17a6-cre mouse has strong red fluorescence, indicating that the neurons projecting to the primary auditory cortex are excitatory neurons.

In summary, MG is the main external projection (non-auditory cortex) brain region of PV-positive neurons in the primary auditory cortex, and the neurons projecting to the primary auditory cortex are excitatory neurons.

Embodiment 6 Specific Activation of MG brain Region Neurons Projecting to the Primary Auditory Cortex Can Produce Antidepressant-Like Effects

### 1. Experimental Method

Chemical genetic technology was used to specifically activate MG neurons projecting to the primary auditory cortex, and its antidepressant effect was detected.

Specifically, C57BL/6J mice were randomly divided into two groups, and both groups were stereotactically microinjected with the retrovirus rAAV2/R-hSyn-Cre-WPRE-hGH-pA in the primary auditory cortex. The virus has a strong affinity with nervous system tissues and can be transported retrogradely along axons to synaptic terminals that receive projections. The retrovirus can infect primary auditory cortical neurons and retrogradely transport along dendrites to synaptic terminals, and the Cre enzyme can be transported across synapses to the upper-level neurons received by the terminals.

As shown in Embodiment 5, the external projection brain region received by the PV-positive neurons of the primary auditory cortex is mainly MG. Further, at the same time as the injection of the retrovirus, the two groups of mice were stereo-localized and micro-injected into the MG brain area with chemical genetic virus rAAV-Ef1a-DIO-hM3D(Gq)-mCherry-WPRE-pA (Article No. PT-0042) (hM3Dq group) and control virus rAAV-Ef1a-DIO-mCherry-WPRE-pA (Article No. PT-0013) (mcherry group) expressing only red fluorescence. 21 days after virus injection, all mice were intraperitoneally injected with CNO 2 mg/kg, and the forced swimming test (FST) was performed 35 min after the intraperitoneal injection. Three days later, CNO was intraperitoneally injected again, and the open field test (OFT) behavioral test was performed 35 min later. Three days later, the social defeat model was established (for the specific experimental process, refer to Embodiment 4).

### 2. Experimental Results

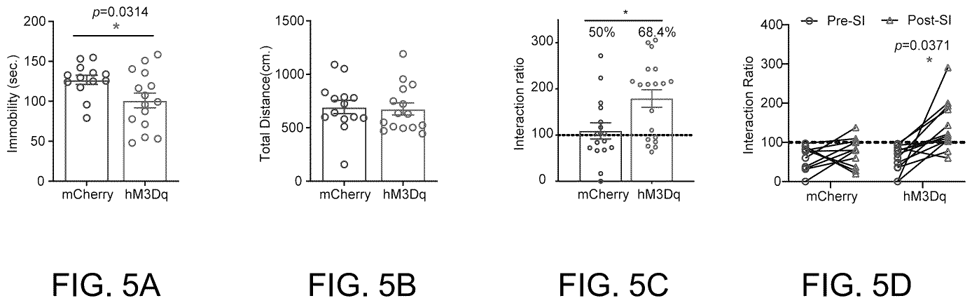

The results are shown in FIG. 5a. Due to the activation of MG neurons that project to the primary auditory cortex, the immobility time of the mice in the hM3Dq group significantly reduced in the forced swimming test. This rapid detection method shows that increased neuronal activity in the MG that projects to the primary auditory cortex has antidepressant effects.

In addition, the results are shown in FIG. 5b. There is no difference in the movement distance (Total distance) of the two groups of mice, indicating that the spontaneous activity of the mice has not changed. That is, this chemical genetic method does not affect spontaneous movement capacity of the organism while antidepressant, and demonstrates that the reduced immobility time in 5a was an antidepressant phenotype rather than a change in motor ability.

FIG. 5c shows that during the social defeat model modeling process, the mcherry group given the control virus and the hM3Dq group given the chemical genetic virus were intraperitoneally injected with CNO once a day in the morning and evening. The social interaction test after modeling showed that the proportion of the non-susceptible group (Interaction Ratio greater than 100) in the normal control mcherry group was 50%, and the proportion of the non-susceptible group in the hM3Dq group was 68.4%, showing an obvious increasing trend, and the total Interaction Ratio of the hM3Dq group was significantly increased compared to the control group.

The above shows that when receiving adverse stimulation and maintaining in a stressful environment, continuous activation of MG neurons that project to the primary auditory cortex can significantly increase the proportion of mice in the depression-non-susceptible group, indicating that under continuous or intermittent frequent stress stimulation environment, activating MG neurons that project to the primary auditory cortex can resist the occurrence of depression after external stimulation.

Pre-SI is the Interaction Ratio measured by the first test after modeling to separate the depressed mice (Interaction Ratio less than 100), and Post-SI is the Interaction Ratio measured 35 minutes after CNO administration. The results in FIG. 5d show that the depressive phenotype of mice in the mcherry group injected with the control virus did not improve after CNO was given, indicating that the control virus injection had no effect and that CNO itself had no antidepressant effect. However, after the hM3Dq group was given CNO to activate MG neurons projecting to the primary auditory cortex, the Interaction Ratio of SS mice in Pre-SI (as Post-SI after CNO injection) significantly improved, and the depressive-like phenotype was significantly improved, indicating that the activation of MG neurons projecting to the primary auditory cortex can significantly reverse the depressive phenotype, making them exhibit an antidepressant phenotype.

The above results show that activating MG neurons that project to the primary auditory cortex has an antidepressant effect; in a continuous or intermittent but long-term stress stimulation environment, activating MG neurons that project to the primary auditory cortex can reduce occurrence of depression ultimately caused by external stress stimulation. Therefore, the method of activating MG neurons that project to the primary auditory cortex can effectively treat mental illnesses such as depression, and its early application before the disease can prevent the occurrence of mental and neurological diseases such as depression.

### Embodiment 7 Specific Activation of PV-Positive Neurons in the Primary Auditory Cortex That Receives Projections From the MG Brain Region Can Produce Antidepressant-Like Effects

### 1. Experimental Method

The LP/FRT recombinase system was combined with the Cre/loxP recombinase system to specifically activate only PV-positive neurons in the primary auditory cortex that receives projections from the MG.

The specific method is as follows:

The primary auditory cortex of PV-cre mice were used to stereotactically microinjected with the chemical genetic virus AAV2/9-hEF1a-fDIO-hM3D(Gq)-mCherry-ER2-WPRE-pA (Article No. S0335-9) (hM3Dq group) depending on Flp enzyme expression or the control virus AAV2/9-hEF1a-fDIO-mCherry-WPRE-pA (Article No. S0553-9) (mcherry group) only expressing red fluorescence. At the same time, the MG brain region of both groups of mice were stereotactically microinjected with virus AAV2/1-CAG-FLEX-Flpo-WPRE-pA (Article No. S0273-9), which can express Cre enzyme-depended Flp enzymes and can realize anterograde transsynaptic projections. The expression of Cre enzymes in PV-cre mice PV-positive neurons can cause the expression of Flp enzymes projecting from MG, thereby causing the expression of hM3D(Gq) protein in the primary auditory cortex. Intraperitoneal injection of CNO activated PV-positive neuronal cells that received MG projections in the primary auditory cortex.

21 days after virus injection, all mice were intraperitoneally injected with CNO 2 mg/kg, and the forced swimming test (FST) was performed 35 min after the intraperitoneal injection. Three days later, CNO was injected intraperitoneally again, and the open field test (OFT) behavioral test was performed 35 min later. Three days later, the social defeat model was established (for the specific experimental process, please refer to the Embodiment 4).

### 2. Experimental Results

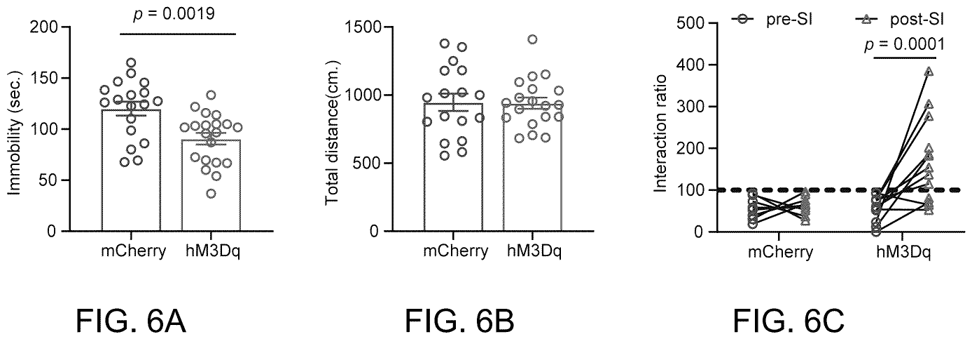

As shown in FIG. 6a, activating PV-positive neurons that receive MG projections in the primary auditory cortex (hM3Dq group) can significantly reduce the immobility time of mice in the forced swimming test. In addition, there was no difference in the total distance (FIG. 6b) in the movement of both groups of mice, indicating that the motor ability of the mice was not affected, that is, this chemical genetic method does not affect the motor ability of the organism while antidepressant. Showing that activation of only the PV in the part of the primary auditory cortex that receives MG projections has significant antidepressant effects.

Pre-SI is the Interaction Ratio measured by the first test after modeling to separate the depressed mice (Interaction Ratio less than 100), and Post-SI is the Interaction Ratio measured 35 minutes after CNO administration. As shown in FIG. 6c, all mice were subjected to the social defeat model after virus injection, and intraperitoneal injection of CNO in the detected SS mice activated the PV-positive neurons in the primary auditory cortex that received MG projections. The depressive phenotype of mice in the control mcherry group after CNO administration was not improved, indicating that injection of control virus had no effect and that CNO itself had no antidepressant effect. However, after the hM3Dq group was given CNO to activate PV-positive neurons in the primary auditory cortex that received MG projections, the Interaction Ratio of SS mice in Pre-SI (as Post-SI after CNO injection) was significantly improved, and the depressive-like phenotype was significantly improved. It shows that activating PV-positive neurons in the primary auditory cortex that receives MG projections can significantly reverse the depressive phenotype and make it exhibit an antidepressant phenotype.

In summary, specific activation of only the PV-positive neurons in the primary auditory cortex that receive MG projections can have an antidepressant effect and can reverse the already formed depressive symptoms. This precise regulation method can greatly reduce the possibility of side effects during clinical application.

### Embodiment 8 Early Antidepressant Individuals Have Reduced Activity of MG Brain Region Neurons Projecting to the Primary Auditory Cortex After Experiencing Stress Stimulation

### 1. Experimental Methods

Furthermore, in order to explore the dynamic changes in neurons in the MG brain region of mice in the social defeat model, C57BL/6J mice were used to stereotactically microinjected with retrovirus rAAV2/R-hSyn-Cre-WPRE-hGH pA (Article No. PT-0136) into the primary auditory cortex.

At the same time, the Cre enzyme-dependent and calcium ion indicator-expressing virus rAAV-Ef1a-DIO-GCaMp6f-WPRE-hGH pA (Article No. PT-0106-9) and the optical fiber plug for optical fiber recording were stereo-injected into the MG. After the virus was injected, it was cleaved by Cre enzyme to express the GCaMP6f calcium indicator protein with green fluorescence. After being excited by the excitation light with a wavelength of 470 nm, its fluorescence signal was collected and recorded by the corresponding program in the computer.

21 days after the virus injection, the 10-day social defeat model was started. While the social defeat model was being established, the activity condition of MG neurons projecting to the primary auditory cortex was recorded.

After 10 days of modeling, the mice were divided into depression-susceptible group (SS) (Interaction Ratio of mice who experienced social defeat was less than 100) and depression-non-susceptible group (RES) (Interaction Ratio of mice who experienced social failure was greater than 100) through social interaction detection.

By analyzing the changes in cell activity of MG neurons projecting to the primary auditory cortex of mice in the depression-susceptible group (SS) and the depression-non-susceptible group (RES) under the three states of modeling (Embodiment 2, FIG. 1d), it can be known that when these neurons experience social defeat stress stimulation, the activity of MG neurons is enhanced or reduced when mice are stimulated by attacking mouse CD1.

### 2. Experimental Results

During the modeling process of the social defeat model, the modeling mice were mainly divided into three states (FIG. 1d as a schematic diagram): CD1 retired breeder mice attacked C57BL/6J mice (Attack); after separated by plexiglass partition plate with air holes, CD1 retired breeder mice approached (tried to approach) C57BL/6J mice (Approach); the CD1 retired breeder mice left C57BL/6J mice (Non-approach).

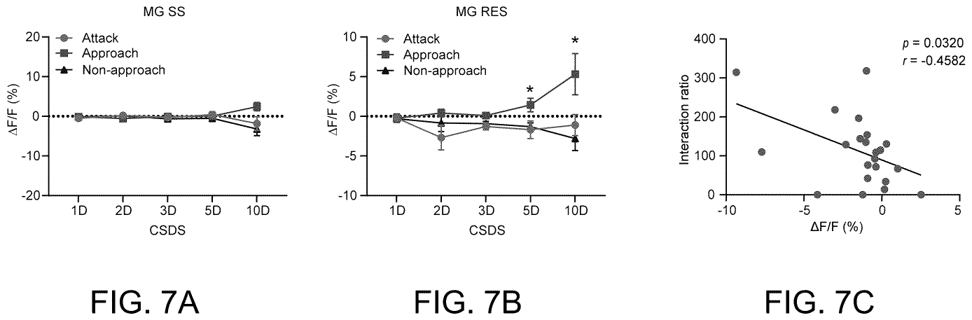

As shown in FIG. 7a, the MG brain region neurons projecting to the primary auditory cortex of SS mice did not fluctuate significantly during the modeling process, indicating that the MG brain region neurons were not involved in the generation process of depression.

As shown in FIG. 7b, the activity of MG neurons in mice of the depression-non-susceptible group (Interaction Ratio greater than 100) decreased when attacked in the early stage (2 days), and the calcium signals of all the mice after modeling when being attacked on the 2nd day were negatively correlated with the Interaction Ratio value after the end of the modeling for 10 days (FIG. 7c). After 5 days of modeling, the approach of CD1 retired breeder mice made the mice feel threatening. In response to this (Approach) stimulation, the activity of MG neurons projecting to the primary auditory cortex of the mice increased significantly and lasted until the end of the modeling on the 10th day (FIG. 7b).

The above results show that when the organism is first exposed to continuous or intermittent but long-term adverse stimulation from the outside world (when stress occurs), it will reduce the activity of neurons in the MG brain region, and further increase the activity of MG neurons in the subsequent continuous stimulation environment (stress response), thereby activating the activity of PV-positive neurons projecting to primary auditory cortex to resist the depressive-like behavior that may be caused by long-term stimulation. Therefore, by simulating this short-term neuronal inhibitory activity, the purpose of enabling the organism to resist stress can be achieved, greatly reducing the occurrence of mental and neurological diseases such as depression.

### Embodiment 9 Short-Term Inhibition of MG Brain Region Neurons Projecting to A1 Can Activate PV-Positive Neurons in A1

### 1. Experimental Method

It can be seen from Embodiments 5 and 6 that by intervening in MG neurons projecting to the primary auditory cortex, PV-positive neurons in the primary auditory cortex can be activated, thereby playing a role of preventing depression occurrence and treating depression. At the same time, Embodiment 8 shows that in individuals with anti-depressant ability, when facing external stimulation in the early stage, the activity of MG neurons will be temporarily inhibited when the organism is stimulated (Attack). This short-term inhibition ability enables mice to increase MG neuron activity in the subsequent confrontation with stress stimulation (Approach), thereby increasing the activity of PV-positive neurons in the primary auditory cortex to achieve an anti-depressant effect.

Optogenetics refers to a technology that combines optical and genetic means to precisely control the activity of specific neurons. The regulation of cell activity by optogenetics depends on the type of light-sensitive channel protein, namely excitatory light-sensitive channel and inhibitory light-sensitive channel. If the channel transferred into the cell is NpHR, when the cell is irradiated with yellow light, the channel opens, a large amount of anions flow in, and hyperpolarization occurs, making it difficult for action potentials to be emitted, thus inhibiting cell activity.

Therefore, the optical genetic method was used, the primary auditory cortex of PV-Cre mice were simultaneously stereotactically microinjected with two viruses, AAV2/2Retro-CAG-FLEX-Flpo-WPRE-pA (Article No. S0273-2/R) for reversely expressing FLP protein and rAAV-Ef1a-DIO-GCaMp6f-WPRE-hGH pA (Article No. PT-0106-9) for expressing calcium ion indicator. At the same time, the virus rAAV-nEF1a-fDIO-eNpHR3.0-mCherry-WPRE-hGH polyA (Article No. PT-1261) for expressing Flp enzyme-dependent inhibitory light-sensitive channel protein was stereotactically injected into the MG. After injection, the virus was cleaved by Flp enzyme to express NpHR3.0 protein, which can inhibit the activity of neurons after it is activated by excitation light with 589nm wavelength.

In order to simulate the attack time of mice in social defeat, the MG brain region of mice was given 10-second light inhibitions for 8 times every day, with a 25-second interval between each stimulation, and a total duration within 5 minutes per day, for three consecutive days, the total inhibition time was 80 seconds per day to inhibit the MG neurons projecting to the primary auditory cortex.

On the fourth day, the mice were placed in a cage of the social defeat model and subjected to a one-day CD1 attack: the mice were first placed in a cage with glass partition plate with air holes and a CD1 retired breeder mouse, and the normal calcium activity of the PV-positive neurons in the primary auditory cortex of the mice was detected when facing the approach of CD1 retired breeder mouse (Pre-approach) without experiencing stimulation. Then the partition plate was removed and the CD1 retired breeder mouse attacked the mouse (Attack). Finally, the partition plate was placed to separate the two mice, and the calcium activity of the PV of Al was recorded when approaching (Approach) separated by the partition plate and when there is no interaction (Non-approach).

### 2. Experimental Results

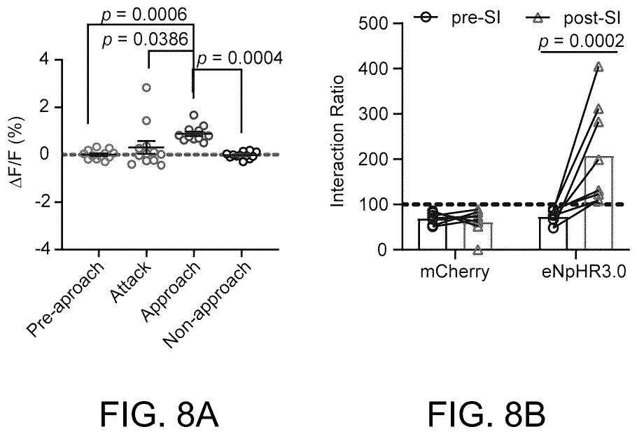

The results in FIG. 8a show that before the mouse was attacked by CD1 retired breeder mouse, there was no obvious change in the activity of PV-positive neurons in the primary auditory cortex when CD1 retired breeder mouse approached, indicating that in a safe situation without external threats, the PV-positive neurons in the primary auditory cortex themselves will not produce obvious excitation or inhibition, and the short-term neuronal inhibition of MG will not produce obvious excitation or inhibition on the PV-positive neurons in the primary auditory cortex.

However, after the mouse was attacked by the CD1 retired breeder mouse, the mouse would be stimulated by the threat of being attacked and produce stress when the CD1 retired breeder mouse approached again. The mouse that had experienced short-term inhibition of MG neurons in advance learned to enhance the activity of PV-positive neurons in the primary auditory cortex under this approach stress. Therefore, after being attacked only once, the activity of PV-positive neurons in the primary auditory cortex of the mouse was rapidly enhanced, which is consistent with the result (FIG. 1e) of PV calcium signal enhancement in RES mice after at least 5 days in Embodiment 2, that is, the mouse has the ability to resist depression when it is first exposed to external stimulation.

The above results show that short-term inhibition of MG neurons projecting to the primary auditory cortex in advance can enable individuals to have the ability of resisting external adverse stimulation in the early stage, thereby reducing depression and other mental and neurological diseases caused by continuous stimulation in the later stage. This short-term preventive measure can effectively reduce the occurrence of mental and neurological diseases in organisms caused by the continuous stress response.

### Embodiment 10 Short-Term Inhibition of MG Neurons Projecting to the Primary Auditory Cortex Can Treat Depression

### 1. Experimental Method

According to the Embodiment 9, before exposure to external stimulation, short-term inhibition of MG neurons projecting to the primary auditory cortex can activate PV-positive neurons in the primary auditory cortex and thus have an anti-depressant effect. Therefore, further research is conducted to determine whether short-term inhibition has a therapeutic effect of reversing depressive-like behavior in individuals who have already developed depression.

C57BL/6J mice were used and randomly divided into two groups. Both groups were stereotactically microinjected with rAAV2/R-hSyn-NLS-FLP-bGH pA (Article No. PT-0133) which expresses Flp protein in reverse into the primary auditory cortex: in the light inhibition group, virus rAAV-nEF1a-fDIO-eNpHR3.0-mCherry-WPRE-hGH polyA (Article No. PT-1261) (eNpHR3.0 group) which expresses Flp enzyme-dependent inhibitory light-sensitive channel protein was injected into the MG at the same time. After virus injection, the virus was cleaved by Flp enzyme to express NpHR3.0 protein, which could inhibit neuronal activity after being activated by excitation light of 589 nm; in the control group, the MG was stereotactically microinjected with the control virus AAV2/9-hEF1a-fDIO-mCherry-WPRE-pA (Article No. S0553-9) (mcherry group) that only expresses red fluorescence.

21 days after virus injection, a 10-day social defeat model was established. The depression-susceptible (SS) mice were obtained by social interaction test. Optical fibers were implanted in the MG brain region of SS mice. After 3 days, 10-second light inhibition was given for 8 times a day, with a 25-second interval between each stimulation, and a total duration within 5 minutes per day for three consecutive days. After light inhibition was finished every day, the mice were put back into the cage with CD1, and CD1 was separated by a glass partition plate with air holes, so that the mice still faced the stimulation of CD1 approaching every day.

After three days of light inhibition, the mice were subjected to social interaction test again to detect the depressive state of the mice.

### 2. Experimental Results

As shown in FIG. 8b, the mice in the mcherry group did not undergo the process of inhibiting MG neurons, and still maintained a phenotype susceptible to depression (Interaction Ratio less than 100), indicating that the depressed mice after modeling can still maintain depressive-like behavior. However, after 3 days of light inhibition of MG neurons projecting to the primary auditory cortex in the eNpHR3.0 group, the social avoidance depressive-like behavior of the mice was significantly reversed. This shows that for individuals who have developed depression, even if they are still exposed to stress stimulation, the method of inhibiting MG neurons projecting to the primary auditory cortex for a short period of less than 14 days (3 days in this embodiment) can produce a significant effect of treating/improving depressive-like behavior.

According to the summary of the above Embodiments 8 to 10: Currently commonly used antidepressant drugs take at least 4 weeks to have effect, and short-term rapid application is 6 to 8 consecutive weeks, followed by long-term use of at least 6 months to maintain the effect. The present invention can quickly make the organism have anti-depressant ability and quickly reverse depressive-like behavior by simulating the inhibition of MG neuron activity caused by short-term stress stimulation of less than 14 days (3 days in this embodiment). This short-term inhibitory intervention method has better acceptance in clinical applications, and patients do not need to take medication for a long time; and for people with high potential risk of illness who have not yet become ill, giving them short-term inhibitory intervention in advance can effectively reduce the occurrence of mental and neurological diseases such as depression in the future.

Finally, it should be noted that the above embodiments only illustrate the technical solutions of the present invention rather than limiting the protection scope of the present invention. For ordinary technicians in the field, other different forms of changes or fluctuations can be made based on the above descriptions and ideas. It is not possible to exhaustively list all implementation methods here. Any modification, equivalent replacement and improvement made within the concept and principle of the present invention should be included in the protection scope claimed in the present invention.

1. A method for preparing a product for diagnosing mental illness and/or neurodegenerative disease, evaluating a risk of mental illness and/or neurodegenerative disease, and/or evaluating therapeutic effect of mental illness and/or neurodegenerative disease, comprising
using a product for detecting activation condition of PV-positive neuronal cells in a primary auditory cortex.

2. A system for evaluating therapeutic effect of mental illness and/or neurodegenerative disease, comprising an information acquisition module, a data processing module and a result output module, wherein:
the information acquisition module is configured to obtain an activation condition of PV-positive neuronal cells in a primary auditory cortex of a patient before and after treatment, or to obtain the activation condition of the PV-positive neuronal cells in the primary auditory cortex of a treatment group and a control group;
the data processing module is configured to compare an activation degree of the PV-positive neuronal cells in the primary auditory cortex before and after treatment, or to compare the activation degree of the PV-positive neuronal cells in the primary auditory cortex of the treatment group and the control group;
the result output module is configured to output the therapeutic effect:
if the activation degree of the PV-positive neuronal cells in the primary auditory cortex after treatment is higher than that of the PV-positive neuronal cells in the primary auditory cortex before treatment, the treatment is effective; otherwise, the treatment is ineffective;
or, if the activation degree of the PV-positive neuronal cells in the primary auditory cortex of the treatment group is higher than that of the PV-positive neuronal cells in the primary auditory cortex of the control group, the treatment is effective; otherwise, the treatment is ineffective.

3. A method for preparing a product for resisting mental illness and/or neurodegenerative disease, comprising
using a product for activating or enhancing activation of PV-positive neuronal cells in a primary auditory cortex or medial geniculate nucleus neurons projecting to the primary auditory cortex or projections from the medial geniculate nucleus to the primary auditory cortex.

4. (canceled)

5. (canceled)

6. The method according to claim 3, wherein the resisting mental illness and/or neurodegenerative disease is a treatment and/or prevention of mental illness and/or neurodegenerative disease.

7. A tangible computer-readable medium encoded with a program, wherein the program, when executed, causes a device to perform a treatment method, and the treatment method comprises at least one of the following methods:
activating or enhancing an activation of PV-positive neuronal cells in a primary auditory cortex;
activating or enhancing an activation of medial geniculate nucleus neurons projecting to the primary auditory cortex;
activating or enhancing an activation of MG projections to the primary auditory cortex; and
taking less than 14 days of a medial geniculate nucleus neuron activity inhibition treatment as a course of treatment;
treating a subject's mental illness and/or neurodegenerative disease or reducing the subject's possibility of developing a mental illness and/or neurodegenerative disease.

8. The computer-readable medium according to claim 7, wherein the device is an instrument that stimulates neurons through nuclear magnetic resonance, sound waves, vibration or electric current.

9. A method for resisting mental illness and/or neurodegenerative disease, or treating and/or preventing depression, wherein the method achieves a purpose of resisting depression through at least one of the following methods:
activating or enhancing an activation of PV-positive neuronal cells in a primary auditory cortex;
activating or enhancing an activation of medial geniculate nucleus neurons projecting to the primary auditory cortex;
activating or enhancing an activation of projections of a medial geniculate nucleus to the primary auditory cortex; and
inhibiting an activity of the medial geniculate nucleus, taking less than 14 days of an activity inhibition treatment as a course of treatment, but not limited to one treatment course.

10. The method according to claim 9, wherein the resisting mental illness and/or neurodegenerative disease is a treatment and/or prevention of mental illness and/or neurodegenerative disease.

11. A method for evaluating therapeutic effect of antidepressant treatment, comprising establishing a social defeat model on experimental animals, randomly dividing the experimental animals into a control group and a treatment group after successful establishment of the model, and carrying out an antidepressant treatment on the experimental animals in the treatment group, and after treatment, comparing an activation degree of PV-positive neuronal cells in a primary auditory cortex of the experimental animals in the control group and the treatment group; if the activation degree of the PV-positive neuron cells in the primary auditory cortex of the treatment group is higher than that of the PV-positive neuron cells in the primary auditory cortex of the control group, the treatment is effective; otherwise, the treatment is ineffective.

12. (canceled)

13. (canceled)

14. The method according to claim 9, wherein the activating or enhancing the activation is performed by using one or more of a transcranial magnetic stimulator, an electroencephalogram recorder, a vibrator, a sound frequency generator, and an electric current stimulator.
